(19) United States
(12) Patent Application Publication
(10) Pub. No.: US 2018/0208901 A1
(43) Pub. Date: Jul. 26, 2018

(54) METHOD FOR FACILITATING FUNCTIONS AND CHARACTERISTICS OF CORNEAL ENDOTHELIAL CELLS

(71) Applicant: Xinyi WU, Jinan Shandong (CN)

(72) Inventors: Xinyi WU, Jinan Shandong (CN); Peng SUN, Jinan Shandong (CN)

(21) Appl. No.: 15/877,361

(22) Filed: Jan. 22, 2018

(30) Foreign Application Priority Data
Jan. 24, 2017 (CN) 201710054356.7

(51) Int. Cl. C12N 5/071; C12N 5/077

(52) U.S. Cl. CPC C12N 5/0692; C12N 5/0653; C12N 2509/00; C12N 2501/113; C12N 2501/11; C12N 2320/32; C12N 2527/00; C12N 2506/1384

(57) ABSTRACT

The present disclosure discloses a method for facilitating functions and characteristics of corneal endothelial cells, comprising the following steps of: separating and culturing human orbital adipose-derived stem cells, and extracting a conditioned culture medium; separating and culturing primary human corneal endothelial cells; adding the conditioned culture medium in a basal culture medium for the human corneal endothelial cells, and culturing and proliferating the human corneal endothelial cells. In the present disclosure, the human corneal endothelial cells cultured by the conditioned culture medium extracted from human orbital adipose-derived stem cells have high adherence and proliferation capacities. Human corneal endothelial cells cultured in vitro can be sub-cultured over 10 generations. The proliferation multiple is higher and the morphology and functions of the human corneal endothelial cells can be maintained. Experiments on animals have proved that the human corneal endothelial cells cultured in vitro have excellent cell repair effects.

### TECHNICAL FIELD

The present disclosure belongs to the technical field of tissue engineering and ophthalmic repairing, and in particular to a method for facilitating functions and characteristics of corneal endothelial cells.

### BACKGROUND

The cornea has five layers, which are successively epithelium, Bowman's membrane, stroma, Descemet's membrane and endothelium from front to rear. The innermost endothelium is very important to maintain the transparency and the normal physiological functions of the cornea. The endothelial cells are monolayer cells each having a height of about 50 μm and a width of about 20 μm. Human corneal endothelial cells (Hcecs) regulate the transparency of the cornea through a pump function and a barrier function. As people get older, the density of corneal endothelial cells gradually becomes lower. When the density of cells is less than 500 to 800 cells/mm², it is likely to result in decompensation of corneal endothelium, and the cornea will lose transparency due to durative edema. Since the corneal endothelial cells of an adult have no proliferation ability, after damaged, the corneal endothelial cells can only be repaired by the extension and migration of cells around the damaged region.

At present, human corneal endothelial cells are optimal seed cells for treating decompensation of corneal endothelium. However, the shortage of corneal donors becomes a global issue. Therefore, corneal endothelial cells are cultured and proliferated in vitro and then used in studies on corneal endothelial cell transplantation, tissue engineering and the like. A common culture method is to tear down the Descemet's membrane and endothelium of the cornea from a donor, extract corneal endothelial cells by enzyme digestion, and culture the corneal endothelial cells in a culture medium which is a basal culture medium added with various growth factors.

However, culturing corneal endothelial cells in vitro has the following disadvantages: the normal morphology and functions of corneal endothelial cells cannot be maintained after they are sub-cultured over multiple generations, so that this method cannot be applied in clinic treatment and related studies. Therefore, at present, it is urgent to find a proper culture method to solve the problem that human corneal endothelial cells cannot be sub-cultured over multiple generations while maintaining the morphology and functions of cells.

### SUMMARY

In view of the problems in the prior art, the present disclosure provides a method which can still maintain the normal morphology of corneal endothelial cells after they are sub-cultured over multiple generations and facilitate the functions and characteristics of corneal endothelial cells. In the present disclosure, first, human orbital adipose-derived stem cells (O-ASCs) are separated and cultured, and a conditioned culture medium is extracted. Then, primary human corneal endothelial cells are extracted, the conditioned culture medium for human orbital adipose-derived stem cells is added in a basal culture medium for human corneal endothelial cells. The results show that the human corneal endothelial cells cultured in vitro by this method can be sub-cultured over 10 generations, and the capacities such as proliferation and repairing of cells can be facilitated while maintaining the normal morphology. Compared with the culture methods in the prior art, unexpected technical effects are achieved.

The present disclosure employs the following technical solutions.

A first objective of the present disclosure is to provide a method for facilitating functions and characteristics of corneal endothelial cells, including the following steps of:

separating and culturing human orbital adipose-derived stem cells (O-ASCs), and extracting a conditioned culture medium;

separating and culturing primary human corneal endothelial cells; and

adding the conditioned culture medium in a basal culture medium for the human corneal endothelial cells, and culturing and proliferating the human corneal endothelial cells.

In the present disclosure, facilitating the functions and characteristics of corneal endothelial cells means facilitating the proliferation and differentiation capacity and the repair capacity of corneal endothelial cells after they are sub-cultured over multiple generations while maintaining the polygonal morphology of the corneal endothelial cells.

Human corneal endothelial cells are developed from neural crest (stem) cells from ectoderm. Also, human orbital adipose-derived stem cells are from neural crest, and have high proliferation capacity and multi-lineage differentiation potential. It is considered that O-ASCs can facilitate the proliferation and functions of corneal endothelial cells by mechanisms such as paracrine (i.e., cell factors in the culture liquid). Therefore, human orbital adipose-derived stem cells are used as the cell source for the conditioned culture medium in the present disclosure. The experimental results show that the conditioned culture medium can effectively facilitate the proliferation and repair capacities of human corneal endothelial cells.

The method for separating and culturing human orbital adipose-derived stem cells includes the following steps of:

collecting human orbital adipose tissues under sterile conditions, washing for several times with PBS, soaking for 30 s with ethanol, washing for several times with PBS again, removing megascopic blood vessels and connective tissues, cutting into particles, adding collagenase digestion solution, and shaking and digesting in a constant-temperature shaker; then, adding a same volume of low-sugar DMEM culture medium containing FBS for neutralization, and centrifuging; discarding supernatant lipid and liquid, re-suspending with sterile PBS, centrifuging, discarding supernatant liquid, adding a proper amount of DMEM culture medium, filtering with a filter screen, mixing uniformly and transferring to a sterile culture dish, and culturing in an incubator containing 5% CO₂ at 37° C.; replacing the culture medium for the first time after 48 to 72 hours, subsequently every 2 to 3 days; and sub-culturing when the cell fusion reaches 80% to 90%.

Preferably, the method for separating and culturing human orbital adipose-derived stem cells includes the following steps of:

collecting human orbital adipose tissues under sterile conditions; under sterile conditions, washing for three times with PBS, soaking for 30 s with 75% ethanol, washing for three times with PBS again, removing megascopic blood vessels and connective tissues, cutting into particles in 1 mm³, adding 2 times in volume of 0.1% collagenase digestion solution I (w/v, g/100 mL), and slowly shaking and digesting for 1 hour in a constant-temperature shaker at 37° C.; then, adding a same volume of low-sugar DMEM culture medium containing 10% fetal bovine serum (FBS) (v/v) for neutralization, and centrifuging at 300×g for 100 minutes; discarding supernatant lipid and liquid, re-suspending with sterile PBS, centrifuging at 300×g for 5 minutes, discarding supernatant liquid, adding a proper amount of DMEM culture medium, filtering with a 100 μm filter screen, mixing uniformly and transferring to a sterile culture dish, and culturing in an incubator containing 5% CO₂ at 37° C.; replacing the culture medium for the first time after 48 to 72 hours, subsequently every 2 to 3 days; and sub-culturing when the cell fusion reaches 80% to 90%.

Preferably, the method for extracting a conditioned culture medium includes the following steps of:

using O-ASCs of the second to tenth generations, discarding the culture medium after the growth rate of O-ASCs reaches 50% to 80%, rinsing once with sterile PBS, adding a DMEN culture medium to continuously culture for 12 to 24 hours, collecting supernatant liquid in the cell culture medium, filtering the collected supernatant liquid by a filter to obtain a conditioned culture medium for human orbital adipose-derived stem cells, and storing at −80° C. for standby.

Preferably, the method for separating and culturing primary human corneal endothelial cells includes the following steps of:

microscopically tearing down the endothelium and Descemet's membrane of the cornea by a pair of forceps, incubating in a basal culture medium in an incubator at 37° C. overnight for stabilization; centrifuging, discarding supernatant liquid, and adding collagenase for digestion; and, separating corneal endothelial cells from the Descemet's membrane by pipetting for multiple times, centrifuging, and discarding the supernatant liquid to obtain primary human corneal endothelial cells.

Specifically, the method for separating and culturing primary human corneal endothelial cells includes the following steps of: microscopically tearing down the endothelium and Descemet's membrane of the cornea by a pair of forceps, incubating in a basal culture medium in an incubator at 37° C. overnight for stabilization; centrifuging at 300×g for 5 minutes; discarding supernatant liquid, and adding 0.1% collagenase I for digestion for 1 to 2 hours at 37° C.; and, separating corneal endothelial cells from the Descemet's membrane by pipetting for multiple times, centrifuging at 400×g for 5 minutes, and discarding the supernatant liquid to obtain primary human corneal endothelial cells.

The method for culturing and proliferating the human corneal endothelial cells includes the following steps of: re-suspending the obtained primary human corneal endothelial cells by a basal culture medium containing the conditioned culture medium, inoculating the cell suspension to a well of a culture plate, and culturing under 5% CO₂ at 37° C.; replacing the culture medium for the first time after 48 hours, subsequently every other day; and sub-culturing at a ratio of 1:2 after the cells are fused.

Specifically, the method for culturing and proliferating the human corneal endothelial cells includes the following steps of:

re-suspending the cells by a basal culture medium containing the conditioned culture medium, inoculating the cell suspension to a well of a 12-well culture plate, pre-coating a cell adhesion reagent (FNCcoatingmix) in the well, and culturing under 5% CO₂ at 37° C.; replacing the culture medium for the first time after 48 hours, subsequently every other day; and sub-culturing the cells at a ratio of 1:2 after the cell fusion reaches 100%.

In the present disclosure, the sub-culturing at a ratio of 1:2 means that primary cells in a cell culture flask are inoculated into two cell culture flasks for sub-culturing.

Experimental results show that the human corneal endothelial cells cultured in the present disclosure do not need to be coated by a cell adhesion reagent in advance during sub-culturing.

A second objective of the present disclosure is to provide human corneal endothelial cells prepared by the method described above.

The human corneal endothelial cells are hexagonal in vivo. The human corneal endothelial cells cultured by the method of the present disclosure are polygonal, approximately hexagonal, when viewed by a phase contrast microscope, approximately the morphology in vivo; and, the cells are densely joined and arranged in a single-layer mosaic pattern. Experiments show that the cells cultured by this method can still maintain their normal cell morphology after they are sub-cultured over 13 generations.

A third objective of the present disclosure is to provide an application of the human corneal endothelial cells in preparing medicines for treating decompensation of corneal endothelium.

The therapeutic method is to inject human corneal endothelial cells cultured in vitro into an anterior chamber of an eye suffering from decompensation of corneal endothelium. Experiments on animals show that, during the treatment of the decompensation of corneal endothelium, the cells cultured by this method have an excellent repair function.

A fourth objective of the present disclosure is to provide a culture medium for culturing corneal endothelial cells, including a basal culture medium for human corneal endothelial cells and the conditioned culture medium having a mass percentage of 10% to 20%, wherein the basal culture medium for human corneal endothelial cells contains Opti-MEM-I, fetal bovine serum (FBS), epidermal growth factor (EGF), ascorbic acid, CaCl₂, chondroitin sulfate and a mixed solution of penicillin and streptomycin.

It has been proved by experiments that, to realize better comprehensive effects of the sub-cultured human corneal endothelial cells, the basal culture medium for human corneal endothelial cells contains Opti-MEM-I, wherein, in the Opti-MEM-I, the volume percentage of FBS is 8% (v/v), the concentration of EGF is 5 ng/mL, the concentration of ascorbic acid is 20 μg/mL, the concentration of CaCl₂ is 200 mg/L, the weight per volume percentage of chondroitin sulfate is 0.08% (w/v, g/100 mL), and the volume percentage of the mixed solution of penicillin and streptomycin is 1% to 1.25% (v/v).

The mixed solution of penicillin and streptomycin contains 10000 μg/mL of penicillin and 10000 μg/mL of streptomycin.

The technical solutions have the following beneficial effects.

In the present disclosure, the conditioned culture medium extracted from human orbital adipose-derived stem cells cultured in vitro is used for culturing human corneal endothelial cells for the first time. Experiments show that the conditioned culture medium can facilitate the proliferation and repair capacities of corneal endothelial cells, thereby providing effective basis for the cell therapy of human corneal endothelial cells. Additionally, the human orbital adipose is easily available, the process of extracting and culturing human orbital adipose-derived stem cells is simple, and a reliable and sufficient cell source can be provided for culturing corneal endothelial cells.

In the present disclosure, the human corneal endothelial cells cultured by the conditioned culture medium extracted from human orbital adipose-derived stem cells can be stably cub-cultured over 13 generations. The proliferation multiple is higher in comparison with the prior art. Moreover, the cells sub-cultured over multiple generations have high adherence and proliferation capacities. In the past, during sub-culturing, the human corneal endothelial cells cultured in vitro need to be coated with a culture medium in advance by substance (e.g., chitosan, FNCCoatingMix and the like) which can facilitate cell adherence. The experimental results show that the human corneal endothelial cells cultured in the present disclosure still have high adherence and proliferation capacities during sub-culturing, without being coated in advance (the cells are inoculated at a density of 4×10⁴ cells/cm² during sub-culturing, and the cell adherence within 24 hours exceeds 50%).

By culturing human corneal endothelial cells by this method of the present disclosure, the normal morphology of the human corneal endothelial cells can be maintained.

The human corneal endothelial cells obtained by this method of the present disclosure can be repetitively frozen for storage.

By the detection of expression related markers of the sub-cultured human corneal endothelial cells by immunofluorescence or other methods, it is found that Na+/K+ATPase, ZO-1 and N-cadherin are all highly expressed.

Experiments on animals show that the human corneal endothelial cells cultured in the present disclosure can treat decompensation of corneal endothelium, and have excellent repair function.

### BRIEF DESCRIPTION OF THE DRAWINGS

FIG. 1 shows the morphology of O-ASC cells and detection of cell-related markers by immunofluorescence by an inverted phase contrast microscope, where A and B show the separation and culturing of O-ASCs, and C shows immunofluorescence (vimentin).

FIG. 2 is a diagram showing the primary culture of Hcecs and the detection of markers thereof, where A to C show the primary culture of Hcecs, D shows the cell aging and deformation of Hcecs (P8), and E to G show detection of cell-related markers by immunofluorescence.

FIG. 3 shows a scratch test (for detecting the proliferation capacity of cells).

FIGS. 4.1 and 4.2 show detection of related makers of sub-cultured Hcecs, where FIG. 4.1 shows the detection by westernblotting and FIG. 4.2 shows the detection by immunofluorescence.

FIGS. 5.1 and 5.2 show the treatment of decompensation of animal corneal endothelium by human corneal endothelial cells cultured in vitro, where FIG. 5.1 shows the treatment of decompensation of rabbit corneal endothelium and FIG. 5.2 shows the treatment of decompensation of monkey corneal endothelium.

FIG. 6 shows living human corneal endothelial cells and human corneal endothelial cells cultured in vitro in the present disclosure.

### DETAILED DESCRIPTION OF THE DISCLOSURE

It is to be noted that the following detailed description is exemplary, only aimed at providing further understanding of the present disclosure. Unless otherwise specified, the technical and scientific terms used herein have meanings the same as the common meanings interpreted by those skilled in the art.

It is to be noted that the terms used herein are merely for describing specific implementations and not intended to limit the exemplary implementations of the present disclosure. As used herein, unless otherwise specified in the context, a singular form also includes a plural form. In addition, it should be understood that the term “contain” and/or “include”, when used in the description, means the presence of features, steps, operations and/or a combination thereof.

### Explanation of Terms

PBS is the abbreviation of a phosphate buffer solution, which is a conventional buffer solution approximate to the physiological conditions of the human body.

The DMEM culture medium is a Dulbecco's modified Eagle's culture medium, including low-sugar DMEM and high-sugar DMEM. It is a conventional basal culture medium.

The Opti-MEM-I is a reduced serum culture medium. It is the modified form of the EMEM basal culture medium and is a medium formed by adding HEPES, sodium bicarbonate, hypoxanthine, thymine, sodium pyruvate, L-glutamine, insulin, transferrin and the like in the EMEM basal culture medium. This culture medium is a conventional reduced serum culture medium.

The materials and reagents used in the present disclosure can be obtained by conventional means. For example, the low-sugar DMEM culture medium is purchased from HyClone; the collagenase I is purchased from Sigma; the cell adherence reagent (FNCcoatingmix) is purchased from Usbio; the Opti-MEM-I is purchased from Gibco; and, the mixed solution of penicillin and streptomycin containing 10000 μg/mL of penicillin and 10000 μg/mL of streptomycin is purchased from Beijing Solarbio Science & Technology Co., Ltd.

### Embodiment 1

1. Separation and Culture of Human Orbital Adipose-Derived Stem Cells, and Extraction of a Conditioned Culture Medium

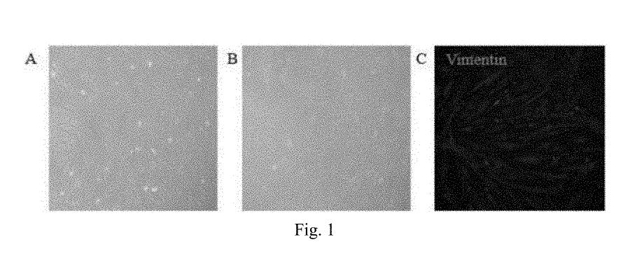

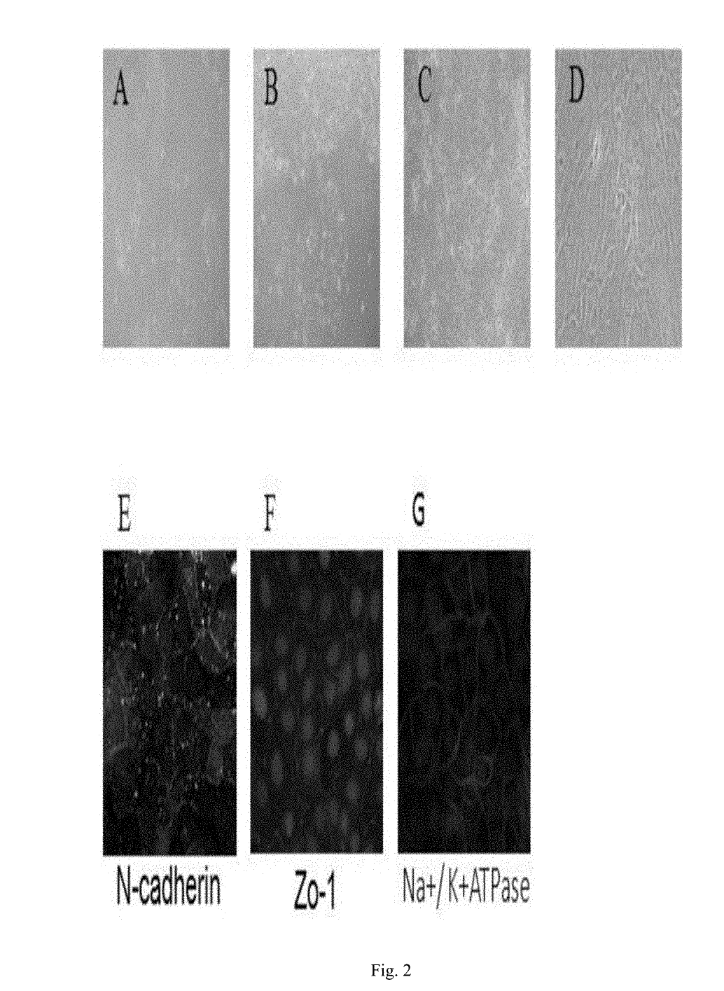

Separation and culture of O-ASCs: the adipose was from a patient who experienced blepharoplasty in the medical cosmetology department of Qilu Hospital. Human orbital adipose tissues were connected under sterile conditions; and under sterile conditions, the human orbital adipose tissues were washed for three times with PBS, soaked for 30 s with ethanol having a volume percentage of 75%, washed for three times with PBS again, removed with megascopic blood vessels and connective tissues, cut into particles in 1 mm³, added with 2 times in volume of 0.1% collagenase digestion solution I, and slowly shaken and digested for 1 hour in a constant-temperature shaker at 37° C. Then, a same volume of low-sugar DMEM culture medium containing 10% FBS was added for neutralization, and the mixture was centrifuged at 300×g for 10 minutes. Supernatant lipid and liquid were discarded; the mixture was re-suspended with sterile PBS and centrifuged at 300×g for 5 minutes; supernatant liquid was discarded; a proper amount of stem cell culture medium was added; and the mixture was filtered with a 100 μm filter screen, mixed uniformly and transferred to a sterile culture dish, and cultured in an incubator containing 5% CO₂ at 37° C. The culture medium was replaced for the first time after 48 to 72 hours and subsequently every 2 to 3 days. Sub-culturing or other experiments were performed when the cell fusion reaches 80% to 90%. The separated and cultured O-ASCs are shown in FIG. 1.

Extraction of the conditioned culture medium: O-ASCs of the second to tenth generations were used, and the culture medium was discarded after the growth rate of O-ASCs reaches 50% to 80%; the O-ASCs were rinsed once with sterile PBS and then added with a fresh stem cell culture medium to continuously culture for 12 to 24 hours; supernatant liquid in the cell culture medium was collected, and the collected supernatant liquid was filtered by a 0.22 μm filter to obtain a conditioned culture medium for O-ASCs; and the conditioned culture medium was stored at −80° C. for standby.

The basal culture medium for human corneal endothelial cells contains Opti-MEM-I and is added with fetal bovine serum (FBS), epidermal growth factor (EGF), ascorbic acid, CaCl₂, chondroitin sulfate and penicillin-streptomycin. In the Opti-MEM-I, the volume percentage of FBS is 8% (v/v), the concentration of EGF is 5 ng/mL, the concentration of ascorbic acid is 20 μg/mL, the concentration of CaCl₂ is 200 mg/L, the weight per volume percentage of chondroitin sulfate is 0.08% (w/v, g/100 mL), and the volume percentage of the mixed solution of penicillin and streptomycin is 1% (v/v).

The proportion of the conditioned culture medium is 10% to 20%.

2. Primary Culture of Human Corneal Endothelial Cells (Hcecs)

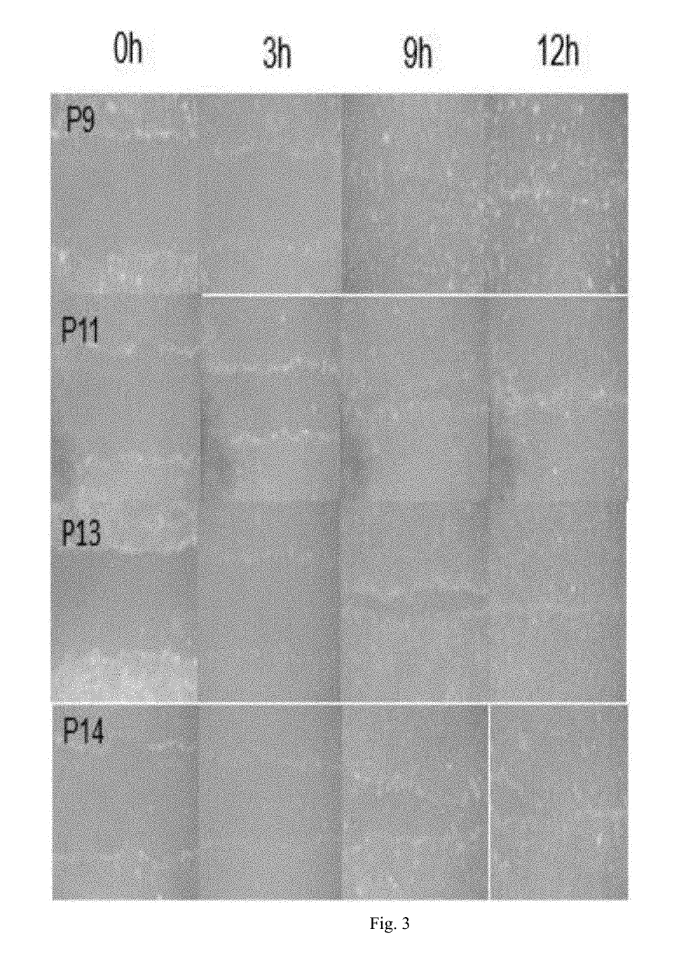

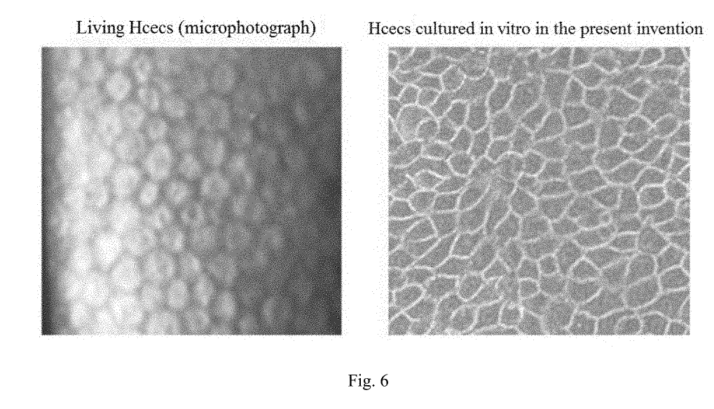

The endothelium and Descemet's membrane of the cornea from the donor were torn down by a pair of forceps, and then incubated in a basal culture medium (Opti-MEM-I, 8% of FBS, 5 ng/mL of EGF, 20 μg/mL of ascorbic acid, 200 mg/L of CaCl₂, 0.08% of chondroitin sulfate and 1% of the mixed solution of penicillin and streptomycin) in an incubator at 37° C. overnight for stabilization. Then, centrifugation was performed at 300×g for 5 minutes. Supernatant liquid was discarded, and 0.1% collagenase I (w/v, g/100 mL) was added for digestion for 1 to 2 hours at 37° C. Corneal endothelial cells were separated from the Descemet's membrane by pipetting for multiple times, and then centrifuged at 400×g at 5 minutes. The supernatant liquid was discarded, and cells were re-suspended by the basal culture medium containing the conditioned culture medium. The cell suspension was inoculated to a well of a 12-well culture plate (which is coated with FNCcoatingmix in advance) and cultured under 5% CO₂ at 37° C. The culture medium was replaced for the first time after 48 hours, and subsequently every other day. After the cell fusion reaches 100%, the cells are sub-cultured at a ratio of 1:2. The primary culture of Hcecs and the detection of markers thereof are shown in FIG. 2. The Hcecs cultured in vitro in the present disclosure are shown in the left picture of FIG. 6. It is observed that the cells are approximately hexagonal. The left picture of FIG. 6 shows living Hcecs which are hexagonal.

3. Functions of Sub-Cultured Hcecs and Detection of Related Markers

Sub-culturing: Hcecs were continuously cultured by the basal culture medium containing the conditioned culture medium from orbital adipose-derived stem cells. The Hcecs were sub-cultured once every 3 to 5 days, at least over 13 generations, while maintaining the polygonal morphology and functions of the cells.

Detection of the proliferation capacity of Hcecs (Hcecs of the ninth, eleventh, thirteenth and fourteenth generations cultured in vitro) by scratch tests: as shown in FIG. 3, Hcecs of the ninth, eleventh and thirteenth generations can completely repair the scratch within 12 hours, and the Hcecs of the fourteenth generation cannot completely repair the scratch within 12 hours. The results show that Hcecs before the fourteenth generation have high proliferation capacity.

Detection of Related Markers:

As a tight junction protein between cells, N-cadherin is expressed in developing corneal endothelial cells.

As a tight junction protein between corneal endothelial cells, ZO-1 is distributed at tight junctions of normal corneal endothelial cells, and is an important constitute of the barrier function of corneal endothelium.

Na+/K+ATPase is presented in the cytoplasm and membrane of normal corneal endothelial cells and is a functional protein essential for the pump function of the corneal endothelial cells.

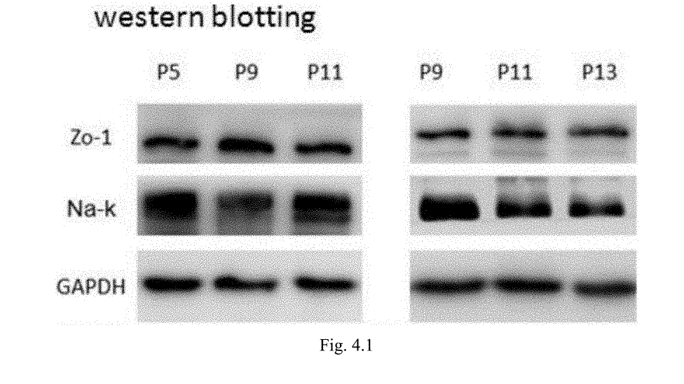

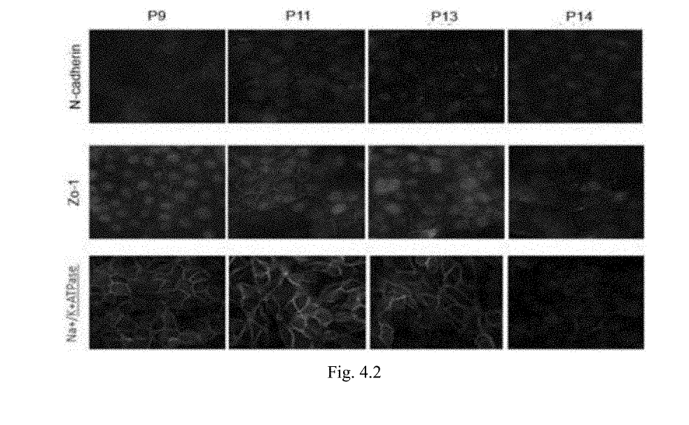

The N-Cadherin, ZO-1 and Na+/K+ATPase were detected by immunofluorescence. The experimental results are shown in FIG. 4.2. Hcecs sub-cultured over multiple generations express the N-Cadherin, ZO-1 and Na+/K+ATPase.

Detection by westernblotting was performed (the cells of the fifth, ninth, eleventh, ninth, eleventh and thirteenth generations, corresponding to P5, P9, P11, P9, P11 and P3 in FIG. 4.1). The experimental results are shown in FIG. 4.1. Hcecs sub-cultured over multiple generations express ZO-1 and Na+/K+ATPase.

4. Verification of the Repair Capacity of Sub-Cultured Hcecs by Experiments on Animals

(1) Modeling of corneal endothelial function insufficiency of New Zealand white rabbits and rhesus monkeys: endothelium was removed by surgery.

(2) Hcec transplantation experiments: Hcecs of the eleventh generation cultured in vitro were injected into the anterior chamber; after the operation, examinations by slit lamps and by anterior segment optical coherence tomography (AS-OCT) were regularly performed as to the change in cornea; and after the operation, histological examination and other examinations were also performed on the cornea.

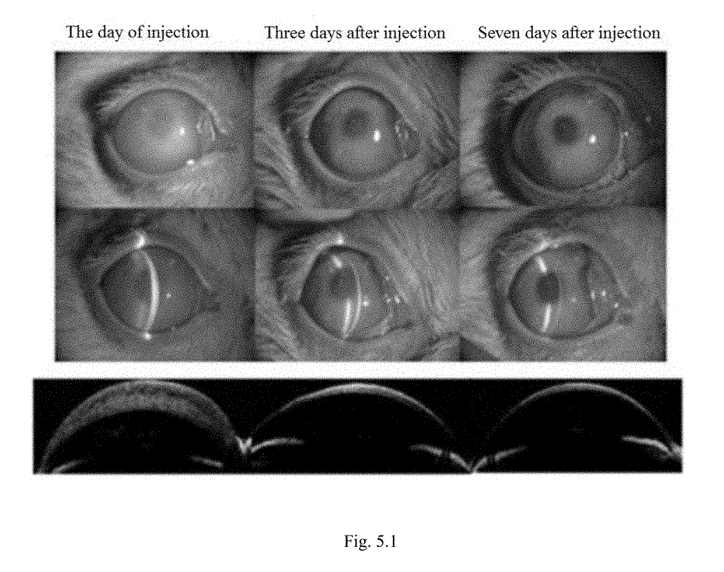

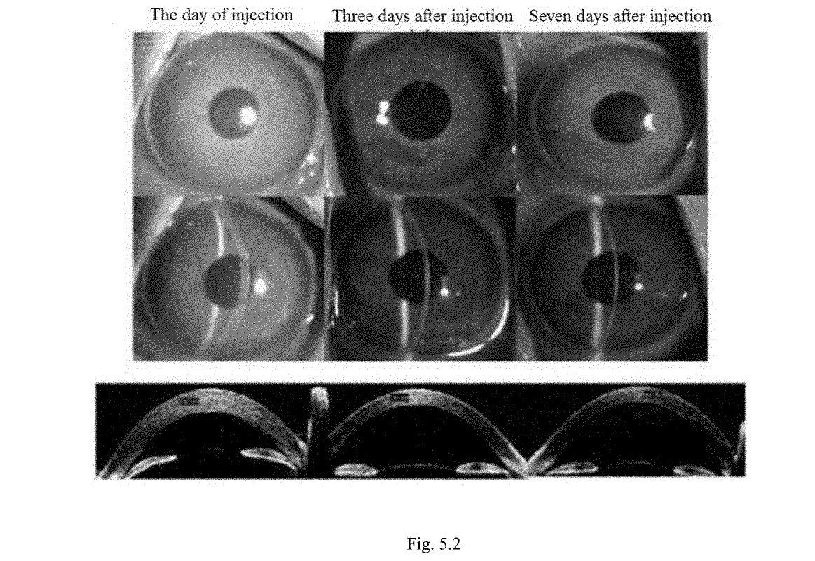

The results of experiments on animals are shown in FIG. 5 (FIG. 5.1 shows the result of experiments on the New Zealand white rabbits, FIG. 5-2 shows the result of experiments on the rhesus monkeys, upper and middle pictures are slit-lamp pictures, and the lower picture is the AS-OCT picture). Models of animals suffering from decompensation of corneal endothelium were established, and Hcecs were then injected into the anterior chamber and observed).

The results show that, as shown in FIG. 5.1, after the treatment of 7 days, by injecting Hcecs into the anterior chamber, the corneal edema and nubecula of the New Zealand white rabbits are gradually alleviated and the cornea finally becomes transparent, and the thickened cornea gradually becomes thinner and finally becomes basically normal.

As shown in FIG. 5.2, after the treatment of 7 days, by injecting Hcecs into the anterior chamber, the corneal edema and nubecula of the rhesus monkeys are gradually alleviated and the cornea finally becomes transparent, and the thickened cornea gradually becomes thinner and finally becomes basically normal.

Conclusion: the Hcecs cultured by the method of the present disclosure can recover the transparent cornea of animals suffering from decompensation of corneal endothelium. The cell therapy effect of Hcecs is fully proved.

The embodiments are merely preferred implementations of the present disclosure, and the implementations of the present disclosure are not limited thereto. Any other alternations, modifications, replacements, combinations and simplifications made without departing from the spirit essence and principle of the present disclosure shall be regarded as equivalent substitutions and shall fall into the protection scope of the present disclosure.

What is claimed is:

1. A method for facilitating functions and characteristics of corneal endothelial cells, comprising following steps:
separating and culturing human orbital adipose-derived stem cells, and extracting a conditioned culture medium;
separating and culturing primary human corneal endothelial cells; and
adding the conditioned culture medium in a basal culture medium for the human corneal endothelial cells, and culturing and proliferating the human corneal endothelial cells.

2. The method according to claim 1, wherein
a method for separating and culturing human orbital adipose-derived stem cells comprises following steps:
collecting human orbital adipose tissues under sterile conditions, washing for several times with PBS, soaking for 30 s with ethanol, washing for several times with PBS again, removing megascopic blood vessels and connective tissues, cutting into particles, adding collagenase digestion solution, and shaking and digesting in a constant-temperature shaker;
then, adding a same volume of low-sugar DMEM culture medium containing FBS for neutralization, and centrifuging;
discarding supernatant lipid and liquid, re-suspending with sterile PBS, centrifuging, discarding supernatant liquid, adding a DMEM culture medium, filtering with a filter screen, mixing uniformly and transferring to a sterile culture dish, and culturing in an incubator containing 5% CO₂ at 37° C.;
replacing the culture medium for the first time after 48 to 72 hours, subsequently every 2 to 3 days; and sub-culturing when the cell fusion reaches 80% to 90%.

3. The method according to claim 1, wherein
a method for extracting a conditioned culture medium comprises following steps:
using O-ASCs of the second to tenth generations, discarding the culture medium after the growth rate of O-ASCs reaches 50% to 80%, rinsing once with sterile PBS, adding a DMEN culture medium to continuously culture for 12 to 24 hours, collecting supernatant liquid in the cell culture medium, filtering the collected supernatant liquid by a filter to obtain a conditioned culture medium for human orbital adipose-derived stem cells, and storing at −80° C. for standby.

4. The method according to claim 1, wherein
a method for separating and culturing primary human corneal endothelial cells comprises following steps of:
microscopically tearing down the endothelium and Descemet's membrane of the cornea by a pair of forceps, incubating in a basal culture medium in an incubator at 37° C. overnight for stabilization;
centrifuging, discarding supernatant liquid, and adding collagenase for digestion; and, separating corneal endothelial cells from the Descemet's membrane by pipetting for multiple times, centrifuging, and discarding the supernatant liquid to obtain primary human corneal endothelial cells.

5. The method according to claim 1, wherein
a method for culturing and proliferating the human corneal endothelial cells comprises the following steps:
re-suspending the obtained primary human corneal endothelial cells by a basal culture medium containing the conditioned culture medium, inoculating the cell suspension to a well of a culture plate, and culturing under 5% CO₂ at 37° C.;
replacing the culture medium for the first time after 48 hours, subsequently every other day; and sub-culturing at a ratio of 1:2 after the cells are fused.

6. Human corneal endothelial cells prepared by the method according to claim 1.

7. The human corneal endothelial cells according to claim 6, wherein
the cells are polygonal, approximately hexagonal, and cells are densely joined and arranged in a single-layer mosaic pattern.

8. An application of the human corneal endothelial cells according to claim 6 in preparing medicines for treating decompensation of corneal endothelium.

9. A culture medium for culturing corneal endothelial cells according to claim 3, comprising a basal culture medium for human corneal endothelial cells and the conditioned culture medium having a mass percentage of 10% to 20%, wherein
the basal culture medium for human corneal endothelial cells contains Opti-MEM-I, fetal bovine serum (FBS), epidermal growth factor (EGF), ascorbic acid, CaCl₂, chondroitin sulfate and a mixed solution of penicillin and streptomycin.

10. The culture medium according to claim 9, wherein
in the Opti-MEM-I, a volume percentage of FBS is 8%, a concentration of EGF is 5 ng/mL, a concentration of ascorbic acid is 20 μg/mL, a concentration of CaCl₂ is 200 mg/L, a weight per volume percentage of chondroitin sulfate is 0.08% (w/v), and a volume percentage of the mixed solution of penicillin and streptomycin is 1% to 1.25%.
